(19) United States
(12) Patent Application Publication
(10) Pub. No.: US 2026/0363973 A1
(43) Pub. Date: Nov. 19, 2026

(54) PROCESS FOR TREATING HUMAN PERSPIRATION AND ASSOCIATED KIT

(71) Applicant: L'OREAL, Paris (FR)

(72) Inventor: Widad AJILI, Chevilly Larue (FR)

(73) Assignee: L'OREAL, Paris (FR)

(21) Appl. No.: 19/133,488

(22) PCT Filed: Nov. 29, 2023
(86) PCT No.: PCT/EP2023/083537

§ 371 Date: May 28, 2025

(30) Foreign Application Priority Data
Nov. 30, 2022 (FR) FR2212563

(51) Int. Cl. A61K 8/24; A61K 8/20; A61K 8/365; A61K 8/73; A61Q 15/00

(52) U.S. Cl. CPC A61K 8/24; A61K 8/20; A61K 8/365; A61K 8/73; A61Q 15/00; A61K 2800/48; A61K 2800/882; A61K 2800/884

(57) ABSTRACT

This invention relates to a cosmetic method for the treatment of human perspiration and optionally body odors resulting from perspiration, as well as an associated cosmetic kit. This process and this kit comprise the use of an aqueous composition A comprising at least magnesium chloride and an aqueous composition B comprising at least dipotassium phosphate, said composition A and/or said composition B comprising at least one anionic thickener, preferably xanthan gum, and/or citric acid.

This invention relates to a cosmetic method for the treatment of human perspiration and optionally body odors resulting from perspiration, as well as an associated cosmetic kit.

This invention in particular relates to a cosmetic method for the treatment of human perspiration and optionally body odors resulting from perspiration, which comprises:
(i) either the mixing just before use of at least one composition A and of at least one composition B, said compositions A and B being packaged separately, followed by the application of the resulting mixture on the surface of the skin;
(ii) or the application on the surface of the skin simultaneously or sequentially of at least one composition A and of at least one composition B packaged separately;
(iii) or the application on the surface of the skin of a composition comprising in the same support at least one composition A and of at least one composition B;
   said composition A comprising in a cosmetically acceptable aqueous medium at least magnesium chloride (MgCl₂);
   said composition B comprising in a cosmetically acceptable aqueous medium at least dipotassium phosphate (K₂HPO₄);
   said composition A and/or said composition B comprising at least one anionic thickener, preferably xanthan gum; and
   said composition A and/or said composition B also comprising at least citric acid.

The underarms as well as certain other parts of the body are generally the place of several discomforts that can stem directly or not from perspiration problems. These phenomena often cause unpleasant and uncomfortable sensations which are primarily due to the presence of sweat resulting from perspiration that can, in certain cases, make the skin moist and wet clothing, in particular at the underarms or the back, as such leaving visible traces. Moreover, the presence of sweat can generate the discharging of body odors that are most of the time unpleasant. Finally, during the evaporation thereof, sweat can also leave behind salts and/or proteins on the surface of the skin which can cause white traces on clothing. Such discomforts are to be taken into account, including in the case of moderate perspiration.

In the cosmetics field, it is as such well known to use a topical application, antiperspirant products containing substances that have for effect to limit and even suppress the sweat flow so as to remedy the problems mentioned hereinabove. These products are in general available in the form of roll-on, sticks, aerosol or spray.

The antiperspirant substances generally consist of aluminum and/or zirconium hydrochlorides. These substances make it possible to reduce the sweat flow by forming a plug on the sweat duct.

However, the use of these substances at high concentrations in order to obtain good effectiveness causes difficulties in terms of the formulation.

In addition, it has been observed that the antiperspirant effectiveness of these substances is limited when they are used alone. This implies that these substances need to be applied several times on the skin in order to obtain a satisfactory effective antiperspirant effect.

Finally, these antiperspirant substances can also leave traces when they are applied which has the consequence of staining clothing.

In order to overcome all of these disadvantages mentioned hereinabove, it has been proposed to seek other effective active substances, that are tolerated by the skin and easy to formulate.

The approaches recently developed include the use of magnesium oxide (MgO) in various dosage forms: this active agent has demonstrated the deodorant effectiveness thereof. However, the formulas containing this active agent have no antiperspirant effect.

A further approach is that described in WO2019/072831: this application describes a cosmetic method for the treatment of human perspiration and optionally body odors resulting from perspiration, which comprises the use of at least one cation Xⁿ⁺ of valency n, of at least one anion Y^{m-} of valency m and of at least one modulating agent. However, not all the cations and the anions described are equivalent, and the dosage form needed to be improved.

There is therefore a real need to implement on the skin an agent intended for the treatment of human perspiration that does not have all of the disadvantages described hereinabove, i.e., that provides a satisfactory antiperspirant effect, in particular in terms of effectiveness and resistance to sweat, and that is tolerated well by the skin. Furthermore, there is a need for such an agent intended for the treatment of human perspiration, that can be readily formulated, is non-irritant, and that is stable during storage.

The Applicant surprisingly discovered that this objective could be achieved using a cosmetic process for the treatment of human perspiration and optionally body odors resulting from perspiration, which comprises the use of at least magnesium chloride (MgCl₂), dipotassium phosphate (K₂HPO₄) and citric acid, in aqueous compositions, at least one of which comprises xanthan gum. Magnesium chloride (MgCl₂) and dipotassium phosphate (K₂HPO₄) are water-soluble salts, non-irritant for the skin. The compositions obtained are easy to formulate, stable, effective and compatible with the pH of the skin.

Therefore, this invention relates to a cosmetic process for the treatment of human perspiration and optionally body odors resulting from perspiration, which comprises the use of at least magnesium chloride (MgCl₂), dipotassium phosphate (K₂HPO₄) and citric acid, in aqueous compositions, at least one of which comprises at least one anionic thickener, preferably xanthan gum.

Magnesium chloride and dipotassium phosphate are formulated in two different compositions, one comprising magnesium chloride (MgCl₂) (composition A), and the other comprising dipotassium phosphate (K₂HPO₄) (composition B). Citric acid (modulating agent), for its part, can be present in composition A and/or in composition B.

This invention therefore relates to a cosmetic process for the treatment of human perspiration and optionally body odors resulting from perspiration, which comprises:
(i) either the mixing just before use of at least one composition A and of at least one composition B, said compositions A and B being packaged separately, followed by the application of the resulting mixture on the surface of the skin;
(ii) or the application on the surface of the skin simultaneously or sequentially of at least one composition A and of at least one composition B packaged separately;
(iii) or the application on the surface of the skin of a composition comprising in the same support at least one composition A and of at least one composition B;
   said composition A comprising in a cosmetically acceptable aqueous medium at least magnesium chloride (MgCl₂);
   said composition B comprising in a cosmetically acceptable aqueous medium at least dipotassium phosphate (K₂HPO₄);
   said composition A and/or said composition B comprising at least one anionic thickener, preferably xanthan gum; and
   said composition A and/or said composition B also comprising at least citric acid.

This invention also relates to a cosmetic kit comprising:
a composition A comprising in a cosmetically acceptable aqueous medium at least magnesium chloride (MgCl₂);
a composition B comprising in a cosmetically acceptable aqueous medium at least dipotassium phosphate (K₂HPO₄);
said composition A and/or said composition B comprising at least one anionic thickener, preferably xanthan gum; and
said composition A and/or said composition B also comprising at least citric acid.

Such a process according to the invention is effective in the treatment of human perspiration. Furthermore, compositions A and B are stable in storage.

“Antiperspirant agent” or “antiperspirant active agent” means any substance or any composition that has the effect of reducing sweat flow and/or reducing the damp sensation on the skin associated with human sweat and/or masking human sweat.

The term “cosmetically acceptable aqueous medium” denotes an aqueous medium compatible with the skin and/or appendages or mucosa thereof, having a pleasant color, odor and texture and not giving rise to unacceptable discomfort (such as tightness), liable to dissuade the consumer from using the composition.

Said cosmetically acceptable medium is also a medium that does not leave any traces during the application thereof, and that does not as such stain clothing.

Said cosmetically acceptable medium is aqueous, i.e., comprises an aqueous phase.

“Sequential” means a successive administration.

“Same support” means that compositions A and B according to the invention are present in the same packaging, in particular in two-compartment packaging that allows for the simultaneous application of compositions A and B.

### Magnesium Chloride (MgCl₂)

Composition A according to the invention comprises in a cosmetically acceptable aqueous medium at least magnesium chloride (MgCl₂). This magnesium chloride (MgCl₂) is a water-soluble salt. In terms of this invention, the term “water-soluble salt” means any salt that, after having been completely dissolved with stirring at 0.5% in a solution of water at a temperature of 25 °C, leads to a solution comprising a quantity of insoluble salt less than 0.05% by weight. Magnesium chloride (MgCl₂) comprises in particular the Mg²⁺ cation.

Composition A comprises an aqueous phase and preferably has a pH between 2 and 6, preferably between 3 and 5. If necessary, the pH is adjusted with a cosmetically acceptable organic or mineral acid or base. Such an acid or such a base are those conventionally used in cosmetics.

Composition A comprises at least magnesium chloride (MgCl₂), and does not contain dipotassium phosphate (K₂HPO₄). Preferably, it does not contain any HPO₄²⁻ anions.

Preferably, composition A comprises magnesium oxide at a content between 5 and 40% by weight relative to the total weight of composition A, preferably between 10 and 30% by weight, preferably between 15 and 25% by weight, preferably between 17 and 22% by weight.

Composition A preferably comprises at least one anionic thickener, preferably xanthan gum.

Composition A can comprise citric acid.

Composition B can comprise sodium citrate. Preferably, composition A comprises from 0.1 to 7% of sodium citrate by weight relative to the total weight of composition A, preferably from 0.5 to 5% by weight, preferably from 1 to 3% by weight.

Preferably, composition A according comprises, in a cosmetically acceptable aqueous medium, by weight relative to the total weight of composition A:
at least 70% by weight of water;
at least magnesium chloride (MgCl₂) at a content between 10 and 30% by weight, preferably between 15 and 25% by weight, preferably between 17 and 22% by weight;
optionally at least one preservative;
between 0.5 and 4% by weight, preferably between 0.6 and 3% by weight, preferably between 0.9 and 2% by weight of at least one anionic thickener, preferably xanthan gum; and
optionally from 0.1 to 7% by weight of sodium citrate, preferably from 0.5 to 5% by weight, preferably from 1 to 3% by weight.

Preferably, composition A comprises from 0.01 to 0.5% of citric acid by weight relative to the total weight of composition A, preferably from 0.015 to 0.05% by weight.

Dipotassium Phosphate (K₂HPO₄)

Composition B comprises in a cosmetically acceptable aqueous medium at least dipotassium phosphate (K₂HPO₄). This dipotassium phosphate (K₂HPO₄) is also a water-soluble salt. Dipotassium phosphate (K₂HPO₄) comprises in particular the HPO₄²⁻ anion.

Composition B comprises an aqueous phase and preferably has a pH between 4 and 10, preferably between 6 and 8. If necessary, the pH is adjusted with a cosmetically acceptable organic or mineral acid or base. Such an acid or such a base are those conventionally used in cosmetics.

Composition B comprises at least dipotassium phosphate (K₂HPO₄), and does not contain magnesium chloride (MgCl₂). Preferably, it does not contain any Mg²⁺ cations.

Preferably, composition B comprises at least dipotassium phosphate (K₂HPO₄) at a content between 5 and 30% by weight relative to the total weight of composition B, preferably between 10 and 25% by weight, preferably between 15 and 20% by weight.

The weight ratio between magnesium chloride and dipotassium phosphate varies preferably from 3:1 to 1:1, preferably from 2:1 to 1:1, and more preferably from 1.5:1 to 1:1.

The total concentration of magnesium chloride and dipotassium phosphate varies preferably from 10 to 50% by weight, preferably between 13 and 40% by weight, and more preferably from 15 to 30% by weight relative to the total weight of compositions A and B.

Composition B preferably comprises at least one anionic thickener, preferably xanthan gum.

Preferably, composition B comprises citric acid.

Preferably, composition B does not contain sodium citrate.

Preferably, composition B comprises, in a cosmetically acceptable aqueous medium, by weight relative to the total weight of composition B:
at least 70% by weight of water;
at least dipotassium phosphate (K₂HPO₄) at a content between 10 and 30% by weight relative to the total weight of composition B, preferably between 10 and 25% by weight, preferably between 15 and 20% by weight;
optionally at least one preservative;
between 0.1 and 7% by weight of citric acid, preferably between 0.5 and 5% by weight, preferably between 1 and 4% by weight; and
between 0.5 and 4% by weight, preferably between 0.6 and 2% by weight, preferably between 0.7 and 1.5% by weight of at least one anionic thickener, preferably xanthan gum.

### Citric Acid

The citric acid present in composition A and/or B is a modulating agent.

Composition A can comprise citric acid. Preferably, composition A comprises from 0.01 to 0.5% of citric acid by weight relative to the total weight of composition A, preferably from 0.015 to 0.05% by weight.

Preferably, citric acid is present in composition B, i.e., with dipotassium phosphate (K₂HPO₄).

Citric acid is present in composition B at a content between 0.1 and 10% by weight relative to the total weight of composition B, preferably between 0.5 and 5% by weight, preferably between 1 and 4% by weight.

Preferably, citric acid is present in composition B, and the weight ratio between citric acid and dipotassium phosphate is between 0.1 and 1, preferably between 0.15 and 0.5.

Preferably, the composition resulting from the mixture of compositions A and B has a pH between 5 and 8, preferably between 5.1 and 6.

### Anionic Thickener

Composition A and/or B comprises at least one anionic thickener, preferably xanthan gum.

Preferably, composition A and composition B each comprise at least one anionic thickener, preferably xanthan gum.

Preferably, the anionic thickener is chosen from natural anionic gums, carrageenans, pectins, phosphated starches and optionally modified carboxyvinyl polymers. Natural anionic gums are in particular xanthan gum, dehydroxanthan and gellan gum.

Preferably, the anionic thickener is chosen from anionic polysaccharide thickeners. More preferably the anionic polysaccharide thickener is chosen from natural anionic gums, carrageenans, pectins and phosphated starches. Natural anionic gums are in particular xanthan gum, dehydroxanthan and gellan gum.

Xanthan is a heteropolysaccharide produced on the industrial scale by aerobic fermentation of the Xanthomonas campestris bacteria. Its structure is composed of a principal chain of β-D-glucoses bonded in β (1,4), similar to cellulose. One glucose molecule out of two carries a lateral trisaccharide chain composed of an a-D-mannose, a β-D-glucuronic acid and a terminal β-D-mannose. The internal residue of mannose is generally acetylated on carbon 6. About 30% of terminal mannose residues carry a pyruvate group bonded in chelated form between carbons 4 and 6. The charged glucuronic acids and pyruvic acids can be ionized, and are therefore responsible for the anionic nature of xanthan (negative charge up to pH equal to 1).

Xanthan gums have a molecular weight between 1,000,000 and 50,000,000.

Xanthan gums are represented for example by the products sold under the trade names Rhodicare by RHODIA CHIMIE, under the trade name SATIAXA E™ by Cargill Texturizing Solutions (for the food, cosmetic and pharmaceutical industry), under the trade name NOVAXAN™ by ADM, and under the trade names Kelzan and Keltrol by CP-Kelco.

The anionic thickener can also be a xanthan derivative, such as dehydroxanthan.

Gellan gum is an anionic linear heteropolysaccharide based on oligosaccharide composed of 4 sugars (tetra-saccharide). D-glucose, L-rhamnose and D-glucuronic acid in 2:1:1 proportions are present in gellan gum in the form of monomer elements.

It is for example sold under the trade name KELCOGEL CG LA by CP KELCO.

Carrageenans are anionic polysaccharides making up the cell walls of various red algae (Rhodophyceae) belonging to the Gigartinaceae, Hypneaceae, Furcellariaceae and Polyideaceae families. They are generally obtained by hot aqueous extraction from natural strains of said algae. These linear polymers, formed by disaccharide structural units, are composed of two D-galactopyranose units bonded alternately by α(1,3) and β(1,4) bonds. They are highly sulfated polysaccharides (20-50%) and the α-D-galactopyranosyl residues can be in 3,6-anhydro form. According to the number and the position of ester-sulfate groups on the repeating disaccharide of the molecule, a distinction is made between several types of carrageenans namely: kappa-carrageenans which has an ester-sulfate group, iota-carrageenans which has two ester-sulfate groups and lambda-carrageenans which have three ester-sulfate groups. The carrageenans are essentially composed of salts of potassium, sodium, magnesium, triethanolamine and/or calcium and polysaccharide sulfate esters.

The carrageenans are in particular marketed by Seppic under the name Solagum®, by Gelymar under the trade name Carragel®, Carralact®, and Carrasol®, by Cargill, under the trade names SATIAGEL and SATIAGUM, and by CP-Kelco under the trade name GENULACTA®, GENUGEL® and GENUVISCO®.

Pectins are linear polymers of α-D-galacturonic acid (at least 65%) bonded at position 1 and 4, with a certain proportion of carboxyl groups esterified with a methanol group. Approximately 20% of the sugars making up the pectin molecule are neutral sugars (L-rhamnose, D-glucose, D-galactose, L-arabinose, D-xylose). L-rhamnose residues are found in all pectins, integrated in the principal chain at positions 1,2. Uronic acid molecules have carboxyl functions. This function provides pectins with the ability to exchange ions, when they are in-COO form.

The anionic thickener can also be a phosphated starch. In particular, monostarch phosphates (of the Am—O—PO—(OX)₂ type), distarch phosphates (of the Am—O—PO—(OX)—O—Am type) or even tristarch phosphates (of the Am—O—PO—(O—Am)₂ type) or mixes thereof can be obtained by crosslinking the starch with phosphorated compounds. Am denotes starch, and X denotes in particular alkaline metals (for example sodium or potassium), alkaline earth metals (for example calcium, magnesium), ammonia salts, amine salts like monoethanolamine, diethanolamine, triethanolamine, amino-3 propanediol-1,2 salts, ammonium salts derived from basic amino acids such as lysine, arginine, sarcosine, ornithine or citrulline. In particular, the phosphated starch is hydroxypropyl starch phosphate, marketed under the name Structure XL by Nouryon.

The optionally modified carboxyvinyl polymers can be copolymers obtained from polymerizing at least one monomer (a) chosen from carboxylic acids with α,β-ethylene unsaturation or esters thereof, with at least one monomer (b) with ethylene unsaturation including a hydrophobic group.

The term “copolymers” denotes both copolymers obtained from two kinds of monomers and those obtained from more than two kinds of monomers such as terpolymers obtained from three kinds of monomers.

In particular, among the optionally modified carboxyvinyl polymers, mention can furthermore be made of sodium polyacrylates such as those marketed under the trade name Cosmedia SP® containing 90% dry matter and 10% water, or Cosmedia SPL® in inverse emulsion containing about 60% dry matter, an oil (hydrogenated polydecene) and a surfactant (PPG-5 Laureth-5), both sold by Cognis. Mention can also be made of partially neutralized sodium polyacrylates, particularly in the form of an inverse emulsion comprising at least one polar oil, for example that sold under the trade name Luvigel® EM by BASF.

The optionally modified carboxyvinyl polymers can also be chosen from crosslinked (meth)acrylic acid homopolymers. The term “(meth)acrylic” in the sense of this application means “acrylic or methacrylic”. For example, mention can be made of those sold by Lubrizol under the trade name Carbopol 910, 934, 940, 941, 934 P, 980, 981, 2984, 5984, Carbopol Ultrez 10 Polymer or by 3V-Sigma under the trade name Synthalen® K, Synthalen® L or Synthalen® M. Among the optionally modified carboxyvinyl polymers, mention can in particular be made of Carbopol (CTFA name: carbomer) and Pemulen (CTFA name: Acrylates/C10-30 akyl acrylate crosspolymer) marketed by Lubrizol.

Preferably, the anionic thickener according to the invention is xanthan gum.

The anionic thickener, preferably xanthan gum, is present in composition A and/or B at a content between 0.1 and 7% by weight relative to the total weight of composition A and/or B, preferably between 0.5 and 5% by weight, preferably between 0.7 and 2% by weight.

Preferably, the anionic thickener, preferably xanthan gum, is present in composition A at a content between 0.3 and 5% by weight relative to the total weight of composition A, preferably between 0.6 and 3% by weight, preferably between 0.9 and 2% by weight.

Preferably, the anionic thickener, preferably xanthan gum, is present in composition B at a content between 0.2 and 4% by weight relative to the total weight of composition B, preferably between 0.6 and 2% by weight, preferably between 0.7 and 1.5% by weight.

### Method of Application

In order to obtain an antiperspirant effect on the skin, according to a first alternative embodiment of the process for the treatment of perspiration of the invention (embodiment (i)), composition A and composition B are packaged separately and are mixed just before use (extemporaneous mixture) then the mixture thus obtained is applied on the surface of the skin to be treated.

According to a second alternative embodiment of the antiperspirant process of the invention (embodiment (ii)), composition A and composition B are packaged separately and applied simultaneously or sequentially on the surface of the skin to be treated. According to this alternative embodiment, when compositions A and B are applied sequentially, the time interval separating the application of composition A from application B can vary from 1 second to 24 hours, more preferably from 10 seconds to 24 hours and even more preferably from 1 minute to 1 hour.

In order to obtain an antiperspirant effect on the skin, according to a third alternative embodiment of the process for the treatment of perspiration of the invention (embodiment (iii)), on the surface of the skin is directly applied a composition comprising in the same support composition A and composition B. In this embodiment (iii), compositions A and B can be for example packaged in a device comprising at least two compartments containing respectively composition A and composition B such as a twin-tubes, a pump bottle with two compartments, an aerosol device comprising two compartments that can include one or more outlet orifices (single-nozzle or double-nozzle), a device provided with a perforated wall such as a grid comprising two compartments; a device comprising two compartments each provided with a ball applicator (multi-ball roll-on); a double-stick.

Preferably, compositions A and B are applied sequentially and even more preferably, composition A (comprising at least magnesium chloride) is applied first.

Preferably, the invention also relates to a process for the cosmetic treatment of human perspiration, and optionally body odors associated with human perspiration, comprising the application on the surface of the skin, in particular on the surface of the armpits, of an effective quantity of composition A and an effective quantity of composition B.

The application time of composition A and/or B on the surface of the skin can vary from 0.5 to 10 seconds, preferably from 1 to 5 seconds. Compositions A and B can be applied several times on the surface of the skin. They can be applied several times, over one day or over several days.

Preferably, the invention also relates to a process for the cosmetic treatment of human perspiration, and optionally body odors associated with human perspiration, comprising a first step of application on the surface of the skin, in particular on the surface of the armpits, of composition A, followed by a second step of application of an effective quantity of composition B.

### Dosage Forms

Compositions A and B can each comprise an aqueous phase.

Compositions A and/or B can independently of one another be presented in any of the dosage forms conventionally used for topical application and particularly in the form of aqueous or hydroalcoholic gels, or in the form of aqueous sticks (i.e., solid).

Preferably, compositions A and B are presented independently of one another in the form of aqueous gels or aqueous sticks (i.e. solid).

They may also, by the addition of an oily or oil phase, be in the form of dispersions such as lotion, emulsions of liquid or semi-liquid consistency such as milk, obtained by dispersing an oily phase in an aqueous phase (O/W) or conversely (W/O), or suspensions or emulsions of soft, semi-solid or solid consistency such as cream or gel, or multiple emulsions (W/O/W or O/W/O), microemulsions, ionic and/or non-ionic type vesicle dispersions, or wax/aqueous phase dispersions. These compositions A and/or B (or the ready-to-use composition or the single anhydrous composition) are prepared according to the usual methods.

Compositions A and/or B may be particularly packaged in pressurized form in an aerosol device or in a pump bottle; packaged in a device equipped with a perforated wall particularly a grid; packaged in a device equipped with a roll-on applicator; packaged in stick form, in loose or compacted powder form. In this respect, they contain the ingredients generally used in this type of products and well-known to a person skilled in the art.

According to another particular embodiment of the invention, compositions A and/or B can be solid in particular in the form of a stick; in loose or compact powder form.

“Solid composition” means that the measurement of the maximum force measured by means of texturometric analysis on inserting a probe in the sample of formula should be at least equal to 0.25 Newton, in particular at least equal to 0.30 Newton, particularly at least equal to 0.35 Newton, assessed under precise measurement conditions as follows: the formulas are poured hot into jars 4 cm in diameter and 3 cm at the bottom. Cooling is performed at ambient temperature. The hardness of the formulas produced is measured after waiting 24 hours. The jars containing the samples are characterized by means of texturometric analysis using a texturometer such as that marketed by Rheo TA-XT2, according to the following protocol: a 5 mm diameter stainless steel ball type probe is brought into contact with the sample at a speed of 1 mm/s. The measurement system detects the interface with the sample with a detection threshold equal to 0.005 Newton. The probe is inserted 0.3 mm into the sample, at a rate of 0.1 mm/s. The measurement apparatus records the progression of the compression force measured over time, during the penetration phase. The hardness of the sample corresponds to the mean of the maximum values of the force detected during penetration, for at least 3 measurements.

Compositions A and/or B can also comprise at least one deodorant agent and/or at least one additional antiperspirant agent.

The term “deodorant agent” denotes in the context of the present invention any agent, alone, with the effect of masking, absorbing, enhancing and/or reducing the unpleasant odor resulting from the decomposition of human sweat.

By way of illustration of these additional deodorant agents can in particular be mentioned bacteriostatic agents or bactericidal agents acting on germs of armpit odor, such as 2,4,4′-trichloro-2′-hydroxydiphenylether (Triclosan), 2,4-dichloro-2′-hydroxydiphenylether, 3′,4′,5′-trichlorosalicylanilide, 1-(3′,4′-dichlorophenyl)-3-(4′-chlorophenyl)urea (Triclocarban) or 3,7,11-trimethyldodeca-2,5,10-trienol (Farnesol); quaternary ammonium salts such as cetyltrimethylammonium salts, cetylpyridinium salts; polyols such as those of the glycerin type, 1,3-propanediol (ZEMEA PROPANEDIOL® sold by Dupont Tate and Lyle Bioproducts), 1,2-decanediol (Symclariol® from Symrise); glycerin derivatives such as for example Caprylic/Capric Glycerides (CAPMUL MCM® from Abitec), Caprylate or glyceryl caprylate (DERMOSOFT GMCY® and DERMOSOFT GMC® respectively from STRAETMANS), Polyglyceryl-2 Caprate (DERMOSOFT DGMC® from STRAETMANS), biguanide derivatives such as polyhexamethylene biguanide salts; chlorhexidine and salts thereof; 4-Phenyl-4,4-dimethyl-2butanol (SYMDEO MPP® from Symrise); cyclodextrins; or alum.

The additional deodorant agents can be present preferably in the compositions according to the invention in weight concentrations ranging from 0.01 to 10% by weight relative to the total weight of the composition.

By way of illustration of additional antiperspirant agents, mention can be made in particular of aluminum and/or zirconium antiperspirant salts or complexes, preferably chosen from aluminum halohydrates; aluminum and zirconium halohydrates, zirconium hydroxychloride and aluminum hydroxychloride complexes with or without an amino acid such as those described in patent U.S. Pat. No. 3,792,068.

Among the aluminum salts, mention can be made in particular of aluminum chlorohydrate in activated or non-activated form, aluminum chlorohydrex, aluminum chlorohydrex polyethyleneglycol complex, aluminum chlorohydrex propyleneglycol complex, aluminum dichlorohydrate, aluminum dichlorohydrex polyethyleneglycol complex, aluminum dichlorohydrex propyleneglycol complex, aluminum sesquichlorohydrate, aluminum sesquichlorohydrex polyethylene glycol complex, aluminum sesquichlorohydrex propyleneglycol complex, aluminum sulfate buffered by aluminum and sodium lactate.

Among the aluminum and zirconium salts, mention can be made in particular of aluminum zirconium octachlorohydrate, aluminum zirconium pentachlorohydrate, aluminum zirconium tetrachlorohydrate, aluminum zirconium trichlorohydrate.

Zirconium hydroxychloride and aluminum hydroxychloride complexes with an amino acid are generally known under the name ZAG (when the amino acid is glycine). Among these products, mention can be made of complexes of aluminum zirconium octachlorohydrex glycine, aluminum zirconium pentachlorohydrex glycine, aluminum zirconium tetrachlorohydrex glycine and aluminum zirconium trichlorohydrex glycine.

Aluminum sesquichlorohydrate is in particular sold under the trade name REACH 301® by SUMMITREHEIS.

Among the aluminum and zirconium salts, mention can be made of the complexes of zirconium hydroxychloride and aluminum hydroxychloride with an amino acid such as glycine having for INCI name: ALUMINUM ZIRCONIUM TETRACHLOROHYDREX GLY for example that sold under the trade name REACH AZP-908-SUF® by SUMMITREHEIS.

Use will be made more particularly of aluminum chlorohydrate in optionally activated form sold under the trade names LOCRON S FLA®, LOCRON P, LOCRON L.ZA by CLARIANT; under the trade names MICRODRY ALUMINUM CHLOROHYDRATE®, MICRO-DRY 323®, CHLORHYDROL 50, REACH 103, REACH 501 by SUMMITREHEIS; under the trade name WESTCHLOR 200® by WESTWOOD; under the trade name ALOXICOLL PF 40® by GUILINI CHEMIE; CLURON 50%® by INDUSTRIA QUIMICA DEL CENTRO; CLOROHIDROXIDO ALUMINIO SO A 50%® by FINQUIMICA. As another antiperspirant agent, mention can be made of the particles of expanded perlite such as those obtained by the method of expansion described in the patent U.S. Pat. No. 5,002,698.

Preferably, compositions A and/or B comprise less than 5% by weight of aluminum salt, preferable less than 3% by weight, preferably less than 1% by weight. Preferably, compositions A and/or B are totally free from aluminum salt.

### Aqueous Phase

Compositions A and/or B can each comprise at least one aqueous phase. They are in particular formulated in aqueous gel.

The aqueous phase of said compositions A and/or B contains water and generally other water-soluble or miscible solvents. The water-soluble or miscible solvents comprise short-chain mono-alcohols for example C₁-C₄ such as ethanol, isopropanol; diols or polyols such as ethyleneglycol, 1,2-propyleneglycol, 1,3-butylene glycol, hexyleneglycol, diethyleneglycol, dipropylene glycol, 2-ethoxyethanol, diethylene glycol monomethylether, triethylene glycol monomethylether and sorbitol.

Preferably, composition A and/or composition B comprises at least 60% by weight of water relative to the total weight of composition A and/or B, preferably at least 70% by weight, preferably at least 75% by weight. Preferably, composition A and/or composition B comprise from 60% to 90% by weight of water relative to the total weight of composition A and/or B, preferably from 70% to 85% by weight, preferably from 75% to 85% by weight.

### Cosmetic Kit

This invention also relates to a cosmetic kit comprising:
a composition A comprising in a cosmetically acceptable aqueous medium at least magnesium chloride (MgCl₂);
a composition B comprising in a cosmetically acceptable aqueous medium at least dipotassium phosphate (K₂HPO₄);
said composition A and/or said composition B comprising at least one anionic thickener, preferably xanthan gum; and
said composition A and/or said composition B also comprising at least citric acid.

Preferably, composition A according comprises, in a cosmetically acceptable aqueous medium, by weight relative to the total weight of composition A:
at least 70% by weight of water;
at least magnesium chloride (MgCl₂) at a content between 10 and 30% by weight, preferably between 15 and 25% by weight, preferably between 17 and 22% by weight;
optionally at least one preservative;
between 0.5 and 4% by weight, preferably between 0.6 and 3% by weight, preferably between 0.9 and 2% by weight of at least one anionic thickener, preferably of xanthan gum; and
optionally from 0.1 to 7% by weight of sodium citrate, preferably from 0.5 to 5% by weight, preferably from 1 to 3% by weight.

Preferably, composition A comprises from 0.01 to 0.5% of citric acid by weight relative to the total weight of composition A, preferably from 0.015 to 0.05% by weight.

Preferably, composition B comprises, in a cosmetically acceptable aqueous medium, by weight relative to the total weight of composition B:
at least 70% by weight of water;
at least dipotassium phosphate (K₂HPO₄) at a content between 10 and 30% by weight relative to the total weight of composition B, preferably between 10 and 25% by weight, preferably between 15 and 20% by weight;
optionally at least one preservative;
between 0.1 and 7% by weight of citric acid, preferably between 0.5 and 5% by weight, preferably between 1 and 4% by weight; and
between 0.5 and 4% by weight, preferably between 0.6 and 2% by weight, preferably between 0.7 and 1.5% by weight of at least one anionic thickener, preferably xanthan gum.

All the features described above, relating to compositions A and B, also apply to the cosmetic kit.

Throughout the description, including the claims, the expression “comprising a” must be understood as being synonymous with “comprising at least one”, unless mentioned otherwise.

The expressions “between . . . and . . . ” and “ranging from . . . to . . . ” are to be understood to be inclusive of the limits, unless specified otherwise.

In the description and the examples, unless mentioned otherwise, the percentages are weight percentages. The percentages are therefore expressed by weight relative to the total weight of the composition (% w/w). The temperature is expressed in degrees Celsius unless mentioned otherwise, and the pressure is the atmospheric pressure, unless mentioned otherwise.

The invention is illustrated in more detail by the non-limiting examples presented hereinafter.

### EXAMPLES

### Example 1: Comparative Compositions

Comparative gel formulas A1 and B1 or B1′ hereinafter were prepared by mixing in water:

TABLE 1

| | Gel A1 | Gel B1 | Gel B1′ |
|---|---|---|---|
| Ingredient | (% w/w) | (% w/w) | (% w/w) |
| Magnesium chloride | 10 | — | — |
| (MgCl₂) | | | |
| Disodium phosphate | — | — | 7.7 |
| (Na₂HPO₄) | | | |
| Dipotassium | — | 9.5 | — |
| phosphate | | | |
| (K₂HPO₄) | | | |
| Citric acid | 1 | — | — |
| Sodium citrate | 3.2 | — | — |
| Hydroxyethylcellulose | 0.75 | 0.75 | 0.75 |
| Preservative | QS | QS | QS |
| Water | QS 100 | QS 100 | QS 100 |

Gels A1, B1 and B1′ based on hydroxyethylcellulose containing 10% MgCl₂ and K₂HPO₄ were formulated. The stabilities of these two gels are not optimal, in particular for the phosphate salt of B1′ which tends to “break” the gel.

The comparative gel B1 is trouble at TO, and unstable after one month at 45 °C.

A comparative gel similar to the gel A1, but which does comprise neither citric acid nor sodium citrate (i.e. only 10% by weight MgCl2, 0.75% hydroxyethylcellulose, a preservative in water), is irritant after 4 days of application with 2 applications/day.

### Example 2: Compositions According to the Invention

Gel formulas A2 and B2 according to the invention hereinafter were prepared by mixing in water:

TABLE 2

| | Gel A2 | Gel B2 |
|---|---|---|
| Ingredient | (% w/w) | (% w/w) |
| Magnesium chloride | 20 | — |
| (MgCl₂) | | |
| Dipotassium phosphate | — | 17.2 |
| (K₂HPO₄) | | |
| Citric acid | — | 3 |
| Xanthan gum | 1 | 0.8 |
| Preservative | QS | QS |
| Water | QS 100 | QS 100 |

### Example 3: Evaluation of Antiperspirant Effectiveness

### AIM

To evaluate the level of antiperspirant effectiveness of the compositions of examples 1 and 2:
4H after the 7th application
24H after the 8th application

### Protocol

### Panel

42 subjects enrolled, 30 included at baseline (20 females, 10 males) aged from 18 to 56 years (mean age: 39 years).
30 subjects completed the study.

### Synopsis

Wash-out period of 21 days without antiperspirant
1 treated armpit versus 1 untreated armpit
8 controlled applications: 2/day for 4 days
Quantity applied: 0.4+/−0.05 g per formula
Procedure: dual application
Apply formula A2 with a finger cot distributing homogeneously on the hairy surface of the armpit, then immediately apply formula B2 in the same way. Drying time in the sitting position for 15 min.
Gravimetric evaluation of the quantity of sweat at Baseline followed by 4H after the 7^{th} application and 24 h after the 8th
Measurement conditions:
   Sweating in sauna at 38 °C and relative humidity of 30 to 40%
   Sweating time: 1 h20 (warmup period: 40 minutes followed by 2×20 min collection periods).

The same study was conducted with comparative compositions A1 and B1; and A1 and B1′.

### Results

1—Antiperspirant Effectiveness 4H after the 7^{th} Application and 24H after the 8^{th} Application According to 2 Types of Calculations

The calculation is based on the ratios (Z) according to the FDA method and estimation of the relative difference:

$$Z = \frac{\frac{PC}{PT}}{\frac{C}{T}} * 100 \qquad [ Math 1 ]$$

Where:
PC=weight of sweat at baseline for untreated armpit (NT)
PT=weight of sweat at baseline for armpit obtained with “Reference” formula mixture
C=weight of sweat TX for untreated armpit
T=weight of sweat TX for armpit obtained with “Reference” formula mixture

### Estimation of Relative Difference in %:

Relative reduction (%)(C−T)*100/C=(1−exp(average of difference))*100 [Math 2]
C=weight of sweat TX for untreated armpit
T=weight of sweat TX for armpit obtained with “Reference” formula mixture

The “Reference” formula mixture is obtained by mixing 2 aqueous formulas A′ and B′ comprising respectively 10% by weight relative to the weight of formula A′ of MgCl₂ and 10% by weight relative to the weight of formula B′ of Na2HPO4.

### Effectiveness Scale:

Very effective: % reduction >20% and over: 50% of the panel show a reduction % greater than 20%
Moderately effective: 10<% reduction <20%
Weakly effective: 0<% reduction <10%.

TABLE 3

| | | | | | Estimation |
|---|---|---|---|---|---|
| | | | sig | | of relative |
| | | | Z | Z | difference (%) |
| Formula | Time | Z % | <80%* | <100%** | 95% CI |
| A2 followed by | 4 H | 70.7 | S | S | 30.6% |
| B2 | | Median | | | [22.1; 38.1] |
| vs armpit NT | 24 H | 64.1 | S | S | 31.2% |
| | | Median | | | [23.4; 38.3] |

*FDA effectiveness
**Europe effectiveness

A substantial and significant reduction of sweating is observed at 4H and at 24H. This demonstrates the effectiveness of the application method of A2 followed by B2, significantly greater than the 20% threshold both at 4 h and at 24H.

### 2—Evolution of Weights of Sweat: Mean Weight+/−95% CI

TABLE 4

| | | 4 H | Delta | |
|---|---|---|---|---|
| | | after 7 | Relative to | |
| | Baseline | applications | initial value | % variation |
| | (Weight | (Weight | (Weight | Relative to |
| Formula | in mg) | in mg) | in mg) | initial value |
| Armpit NT | 745 | 858 | 113 | 14% |
| A2 followed | 758 | 644 | −114 | −21% |
| by B2 | | | | |
| Delta product- | 13 | −214 | −227 | |
| Armpit NT | | | | |

TABLE 5

| | | 24 H | Delta | |
|---|---|---|---|---|
| | | after 8 | Relative to | |
| | Baseline | applications | initial value | % variation |
| | (Weight | (Weight | (Weight | Relative to |
| Formula | in mg) | in mg) | in mg) | initial value |
| Armpit NT | 745 | 860 | 115 | 14% |
| A2 followed | 758 | 609 | −149 | −21% |
| by B2 | | | | |
| Delta product- | 13 | −252 | −264 | |
| Armpit NT | | | | |

The deltas at Baseline are low.

Compared to Baseline, the quantities of sweat increase significantly for the untreated armpit. No significant evolution is observed for the armpit treated with the combination of the invention.

### 3—Bayesian Analysis: Probability for Repeatability of Effectiveness on Another Study

This analysis makes it possible to calculate a probability of obtaining the same result during a repeatability study.

4 performance classes were identified:
Zero effect: % reduction <0%
Slight effect: 0<% reduction <10%
Moderate effect: 10<% reduction <20
Substantial effect: % reduction >20

The probability calculation was performed for each of the classes.

TABLE 6

| | High | Moderate | Low | Zero |
|---|---|---|---|---|
| | P(Δ > | P(10% < Δ < | P(0% < Δ < | P(Δ < |
| Performance | 20%) | 20%) | 10%) | 0%) |
| Probability | 99 | 1 | 0 | 0 |
| 4 h (%) | | | | |
| Probability | 100 | 0 | 0 | 0 |
| 24 h (%) | | | | |

There is 99% to 100% likelihood of obtaining a result greater than 20% reduction at 4 h and at 24 h.

### 4—Positioning of A2 and B2 Versus Compositions A1 and B1, and A1 and B1′

TABLE 7

| | Relative difference (%) | | |
|---|---|---|---|
| Formulas | 4 H | 24 H | sig |
| A1 followed | 13% | 9% | s |
| by B1′ | | | |
| (comparative) | | | |
| A1 followed | 11% | 13% | S |
| by B1 | | | |
| (comparative) | | | |
| A2 followed | 30% | 31% | S |
| by B2 | | | |
| (invention) | | | |

### CONCLUSION

The effectiveness results obtained of 11% reduction in sweat at 4 h and 13% at 24 h for A1 followed by B1 (comparative) are equivalent to those of A1 followed by B1′ (comparative).

On the other hand, under the study conditions, the association according to the invention (A2 followed by B2) shows a substantial antiperspirant effectiveness in gravimetry, significantly greater than the 20% threshold 4 h after 7 applications and 24 h after 8 applications.

The clinical effectiveness observed for this association is of the same level as compositions comprising 15% aluminum salts, with an unprecedented result of 30% at 4 h and 31% at 24 h.

### Example 4: Compositions According to the Invention

Gel formulas A3 and B3 according to the invention hereinafter were prepared by mixing in water:

TABLE 8

| | Gel A3 | Gel B3 |
|---|---|---|
| Ingredient | (% w/w) | (% w/w) |
| Magnesium chloride | 20 | — |
| (MgCl₂) | | |
| Dipotassium phosphate | — | 17.2 |
| (K₂HPO₄) | | |
| Sodium citrate | 1.44 | — |
| Citric acid | 0.02 | 3 |
| Xanthan gum | 1 | 0.8 |
| Preservative | QS | QS |
| Water | QS 100 | QS 100 |

1. A cosmetic process for the treatment of human perspiration and optionally body odors resulting from the perspiration, which comprises:
(i) either the mixing just before use of at least one composition A and of at least one composition B, said compositions A and B being packaged separately, followed by the application of the resulting mixture on the surface of the skin;
(ii) or the application on the surface of the skin simultaneously or sequentially of at least one composition A and of at least one composition B packaged separately;
(iii) or the application on the surface of the skin of a composition comprising in the same support at least one composition A and of at least one composition B;
said composition A comprising in a cosmetically acceptable aqueous medium at least magnesium chloride (MgCl₂);
said composition B comprising in a cosmetically acceptable aqueous medium at least dipotassium phosphate (K₂HPO₄);
said composition A and/or said composition B comprising at least one anionic thickener; and
said composition A and/or said composition B also comprising at least citric acid.

2. The cosmetic process according to claim 1, characterized in that the anionic thickener is chosen from natural anionic gums, carrageenans, pectins, phosphated starches and optionally modified carboxyvinyl polymers.

3. The cosmetic process according to claim 1, characterized in that the anionic thickener is xanthan gum.

4. The cosmetic process according to claim 1, characterized in that composition A comprises the anionic thickener.

5. The cosmetic process according to claim 1, characterized in that composition B comprises at least one anionic thickener.

6. The cosmetic process according to claim 1, characterized in that citric acid is present in composition B at a content between 0.1 and 10% by weight relative to the total weight of composition B.

7. The cosmetic process according to claim 1, characterized in that citric acid is present in composition B, and the weight ratio between citric acid and dipotassium phosphate is between 0.1 and 1.

8. The cosmetic process according to claim 1, which comprises a first step of application on the surface of the skin of composition A, followed by a second step of application of an effective quantity of composition B.

9. A cosmetic kit comprising:
a composition A comprising in a cosmetically acceptable aqueous medium at least magnesium chloride (MgCl₂);
a composition B comprising in a cosmetically acceptable aqueous medium at least dipotassium phosphate (K₂HPO₄);
said composition A and/or said composition B comprising at least one anionic thickener; and
said composition A and/or said composition B also comprising at least citric acid.

10. The kit according to claim 9, characterized in that composition A comprises magnesium oxide at a content between 5 and 40% by weight relative to the total weight of composition A.

11. The kit according to claim 9, characterized in that composition B comprises dipotassium phosphate at a content between 5 and 30% by weight relative to the total weight of composition B.

12. The kit according to claim 9, characterized in that the weight ratio between magnesium chloride and dipotassium phosphate varies from 3:1 to 1:1.

13. The kit according to claim 9, characterized in that the total concentration of magnesium chloride and dipotassium phosphate varies from 10 to 50% by weight, relative to the total weight of compositions A and B.

14. The kit according to claim 9, characterized in that composition A comprises, in a cosmetically acceptable aqueous medium, by weight relative to the total weight of composition A:
at least 70% by weight of water;
at least magnesium chloride at a content between 10 and 30% by weight;
optionally at least one preservative;
between 0.5 and 4% by weight of at least one anionic thickener; and
optionally from 0.1 to 7% by weight of sodium citrate.

15. The kit according to claim 9, characterized in that composition B comprises, in a cosmetically acceptable aqueous medium, by weight relative to the total weight of composition B:
at least 70% by weight of water;
at least dipotassium phosphate (K₂HPO₄) at a content between 10 and 30% by weight relative to the total weight of composition B;
optionally at least one preservative;
between 0.1 and 7% by weight of citric acid; and
between 0.5 and 4% by weight of at least one anionic thickener.

16. The kit according to claim 9, characterized in that compositions A and/or B are presented in the form of aqueous or hydroalcoholic gels, or in the form of aqueous sticks.

17. The kit according to claim 9, characterized in that composition A and/or composition B comprise at least 60% by weight of water relative to the total weight of composition A and/or B.

18. The kit according to claim 9, characterized in that compositions A and/or B comprise less than 5% by weight of aluminum salt.

19. The cosmetic process according to claim 2, characterized in that the anionic thickener is xanthan gum.

20. The cosmetic process according to claim 2, characterized in that composition A comprises the anionic thickener.
